(19) United States
(12) Patent Application Publication
(10) Pub. No.: US 2023/0271036 A1
(43) Pub. Date: Aug. 31, 2023

(54) Fiber Material Composite Having a Region that Neutralizes Reactive Oxygen Species

(71) Applicant: Siemens Aktiengesellschaft, München (DE)

(72) Inventors: Jens Dahl Jensen, Berlin (DE); Ursus Krüger, Berlin (DE); Gabriele Winkler, Berlin (DE); Ana-Isabel Rueda, München (DE)

(73) Assignee: Siemens Aktiengesellschaft, München (DE)

(21) Appl. No.: 18/007,333

(22) PCT Filed: Jul. 15, 2021
(86) PCT No.: PCT/EP2021/069770

§ 371 Date: Jan. 30, 2023

(30) Foreign Application Priority Data
Jul. 31, 2020 (EP) 20188831.0

(51) Int. Cl. A62B 23/02; A41D 13/11; B01D 39/10; B01D 39/16; B01D 39/08; B32B 5/02; B32B 5/26; B32B 15/02; B32B 15/14

(52) U.S. Cl. CPC A62B 23/025; A41D 13/1192; B01D 39/10; B01D 39/1623; B01D 39/08; B32B 5/022; B32B 5/024; B32B 5/266; B32B 5/275; B32B 15/02; B32B 15/14; B01D 2239/0241; B01D 2239/0442; B01D 2239/0428; B01D 2239/0478; B01D 2239/065; B01D 2239/0613; B01D 2239/0618; B01D 2239/0407; B32B 2250/20; B32B 2255/02; B32B 2262/0253; B32B 2307/724; B32B 2571/00

(57) ABSTRACT

Various embodiments of the teachings herein include a fiber material composite. The composite may include: a first region including metallic silver and manganese(IV) oxide for producing reactive oxygen species; and a second region configured to neutralize the reactive oxygen species.

### CROSS-REFERENCE TO RELATED APPLICATIONS

This application is a U.S. National Stage Application of International Application No. PCT/EP2021/069770 filed Jul. 15, 2021, which designates the United States of America, and claims priority to EP Application No. 20188831.0 filed Jul. 31, 2020, the contents of which are hereby incorporated by reference in their entirety.

### TECHNICAL FIELD

The present disclosure relates to fiber materials. Various embodiments of the teachings herein include fiber composites, mouth/nose protectors, and/or items of personal protective equipment.

### BACKGROUND

By way of example, such a fiber material composite can be used in antibacterial and/or antiviral filters or surgical mouth/nose protection masks. Furthermore, the fiber material can be used in filters for respiratory protection devices or in air-conditioning systems exposed to changing ambient temperatures. Application DE 10 2020 203 783.3 describes a fiber material for antibacterial and/or antiviral use that comprises fibers comprising metallic silver and manganese(IV) oxide. This fiber material is configured for example for use for a gas-permeable filter and/or mouth/nose protector. It has been found here that the silver and the manganese(IV) oxide can be applied to fibers very well, and form so-called reactive oxygen species, also known as ROS, with the, actually undesired, moisture from the respiratory air or ambient air.

### SUMMARY

The teachings of the present disclosure include fiber material composites, mouth/nose protectors, and items of personal protective equipment. For example, some embodiments include a fiber material composite (10) having at least a first region (14) that comprises metallic silver and manganese(IV) oxide for producing reactive oxygen species (ROS) and a second region (16) that is configured to neutralize the reactive oxygen species (ROS).

In some embodiments, the first region (14) and/or the second region (16) are in each case configured as a separate ply.

In some embodiments, there is an intermediate region (15) that is configured in particular as a nonwoven.

In some embodiments, the first region (14) comprises manganese dioxide-doped silver particles.

In some embodiments, the first region (14) and/or the second region (16) are in each case configured as a nonwoven, in particular a polypropylene nonwoven.

In some embodiments, the second region (16) has an electrically conducting coating.

In some embodiments, the second region (16) comprises an electrically conducting material, in particular an electrically conducting woven fabric.

In some embodiments, the second region (16) comprises a stainless steel mesh or stainless steel grid.

As another example, some embodiments include a mouth/nose protector (50), comprising a fiber material composite (10) as described herein.

In some embodiments, the second region (16) is arranged such that it is arranged between a mouth region (120) of a wearer (100) of the mouth/nose protector (50) and the first region (14).

In some embodiments, the first region (14) is arranged as the outermost layer on a side that faces away from the mouth region (120) of a wearer (100) of the mouth/nose protector (50).

In some embodiments, there is a hydrophobic layer (12).

In some embodiments, there is a hygroscopic layer.

In some embodiments, the second region (16) is part of the mouth/nose protector (50) and the first region (14) is designed to be exchangeable.

As another example, some embodiments include an item of personal protective equipment comprising a fiber material composite (10) as described herein, wherein a second region (16) of the fiber material composite (10) is arranged between a skin region of a wearer (100) and a first region (14).

### BRIEF DESCRIPTION OF THE DRAWINGS

The teachings of the present disclosure is described and explained in more detail in the text that follows on the basis of the exemplary embodiments illustrated in the figures, in which:

FIG. 1 shows a schematic cross section of a fiber material composite incorporating teachings of the present disclosure;

FIG. 2 shows a schematic cross section of a further fiber material composite incorporating teachings of the present disclosure;

FIG. 3 shows an embodiment of a mouth/nose protector incorporating teachings of the present disclosure; and

FIG. 4 shows a further embodiment of a mouth/nose protector incorporating teachings of the present disclosure.

### DETAILED DESCRIPTION

In some embodiments of the teachings herein, a fiber material composite has at least a first region that comprises metallic silver and manganese(IV) oxide for producing reactive oxygen species and a second region that is configured to neutralize the reactive oxygen species. The first region that forms reactive oxygen species is configured here according to the material combination of metallic silver and manganese(IV) oxide that is mentioned at the outset. The oxygen species here have antiviral and antibacterial properties and should therefore not come into contact with or be taken in by a wearer of a mask in relatively large quantities. To this end, a second region that neutralizes the remaining reactive oxygen species is proposed. This increases the compatibility of the fiber material. The neutralization of the reactive oxygen species may be realized here for example by way of an exchange of electrons between the second region and the reactive oxygen species. This may in particular be realized using an electrically conducting material or a material that enables an exchange of electrons between the reactive oxygen species and the second region.

In some embodiments, the first region and/or the second region is in each case configured as a separate ply. This may result in simpler manufacturing of the regions, by providing for example as a web material a fiber material that produces reactive oxygen species and, separately therefrom, a material that neutralizes reactive oxygen species. The two materials may then be joined, for example adhesively bonded, welded or sewn, to form a composite.

In some embodiments, the fiber material composite has an intermediate region that is configured in particular as a nonwoven. The intermediate region is arranged between the first and the second region and separates them from one another. The antiviral and antibacterial effect of the region that produces reactive oxygen species may thus be optimized, while the remaining ROS that penetrate through the intermediate region may be neutralized in the second region. Furthermore, the intermediate region may have an activated carbon layer that already neutralizes parts of the reactive oxygen species.

In some embodiments, the first region comprises silver and manganese dioxide, in particular manganese dioxide-doped silver particles. It has proven to be particularly efficient to equip a fiber material composite with this material combination since they have high availability and high compatibility.

In some embodiments, the first region and/or the second region is in each case configured as a nonwoven, in particular a polypropylene nonwoven. The first region and the second region may be applied here to a single continuous nonwoven on respectively opposite sides.

In some embodiments, the second region has an electrically conducting coating. It is thus possible to use a coated fiber material for the second region and to use a reactive oxygen species-neutralizing effect of the electrically conducting coating in an ideal manner. The electrically conducting property of the coating here neutralizes the reactive oxygen species by exchanging electrons with the reactive oxygen species.

In some embodiments, the second region comprises an electrically conducting material. A mesh or a woven fabric made of the electrically conducting material may thus be used. In some embodiments, use may be made of a thin, electrically conducting film through which respiratory air can flow and which has low breathing resistance. In some embodiments, the second region may comprise an electrically conducting material in addition to further materials, for example a skin-compatible nonwoven layer.

In some embodiments, the second region comprises a stainless steel mesh or stainless steel grid. It has proven to be particularly advantageous to use a stainless steel mesh as reactive oxygen species-neutralizing region. Using a stainless steel has the advantage that it is highly compatible and has a very good neutralizing effect with respect to the reactive oxygen species. The effect mechanism is based here also on an exchange of electrons.

In some embodiments, there is a mouth/nose protector, in particular by a surgical mask, comprising a fiber material composite incorporating teachings of the present disclosure. The mouth/nose protector thus comprises a fiber material composite having at least a first region that is configured to produce reactive oxygen species and a second region that is configured to neutralize the reactive oxygen species. The first region may be arranged here on the side that faces away from the wearer of the mouth/nose protector.

In some embodiments, the second region is arranged such that it is arranged between a mouth region of a wearer of the mouth/nose protector and the first region. This arrangement ensures that the respiratory air of the wearer contains no or only very small amounts of reactive oxygen species.

In some embodiments, the first region is arranged as the outermost layer on a side that faces away from the mouth region of a wearer of the mouth/nose protector.

In some embodiments, the mouth/nose protector has a hydrophobic layer; this may be configured in particular as the outer layer of the mouth/nose protector that faces away from the wearer. Such a layer is also conceivable between the first region and second region so that the moisture stays in the first region, which can enhance the effect of generating reactive oxygen species.

In some embodiments, the mouth/nose protector has a hygroscopic layer. This may be arranged in the vicinity of the first region so that the moisture can enhance the reactive oxygen species-generating effect.

In some embodiments, the mouth/nose protector is of multi-part design, so that the second region is part of the mouth/nose protector and the first region is designed to be exchangeable. It is thus for example possible for an easy-to-clean electrically conducting second region 16 made of stainless steel to be part of the mouth/nose protector, or even to be designed as a reusable basic element of the mouth/nose protector, and for the first region to be arranged on an exchangeable filter nonwoven that is in turn readily disposable.

Some embodiments include personal protective equipment comprising a fiber material composite incorporating teachings of the present disclosure. The second region is arranged here between a skin region of a wearer and the first region. The antiviral and antibacterial effect may thus also be utilized in an item of clothing, for example a laboratory/doctor's coat.

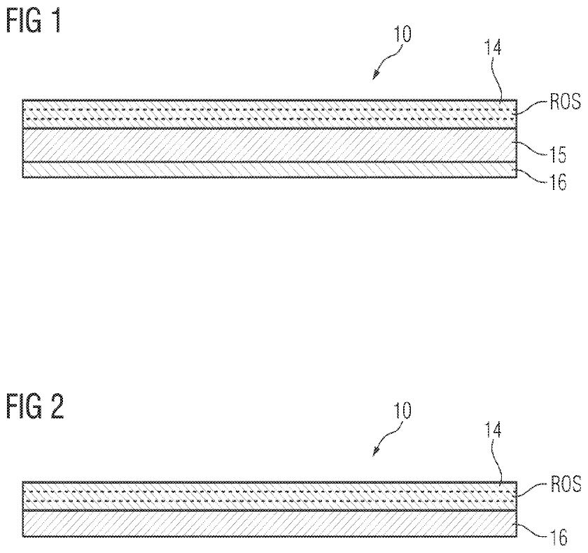

FIG. 1 shows a cross section through a fiber material composite 10. Said composite has a first region 14, a second region 16 and an intermediate region 15 lying between the first and second region 14, 16.

It is schematically indicated in the first region 14 that reactive oxygen species ROS have already been formed in the first region, which have accumulated there and which have an antiviral and antibacterial effect. It is conceivable that all regions 14, 15, 16 are realized by a single nonwoven and are provided from the respective sides with layers that assume the functions of the first region 14 and of the second region 16. The first region 14 may therefore be provided with a silver coating having manganese dioxide doping, and the second region 16 with an electrically conductive coating. It is likewise conceivable that stainless steel fibers/threads are woven into the nonwoven in the second region 16.

In some embodiments, the regions 14, 15, 16 are realized as three separate plies or at least as two plies, i.e. one ply of nonwoven for the first region 14 provided with a material complex that produces reactive oxygen species ROS, one ply of nonwoven for the intermediate region 15 that may be configured as a nonwoven or as a nonwoven with activated carbon, and one ply of electrically conducting woven fabric for the second region 16.

FIG. 2 shows a cross section through an example embodiment of a fiber material composite 1. Here, the first region 14 and the second region 16 directly adjoin one another. This can be achieved either by directly connecting separate plies for each of the regions 14, 16 or by applying/introducing the regions 14, 16 onto/into a single nonwoven. The embodiment thus shown is more compact and may have relatively low breathing resistance.

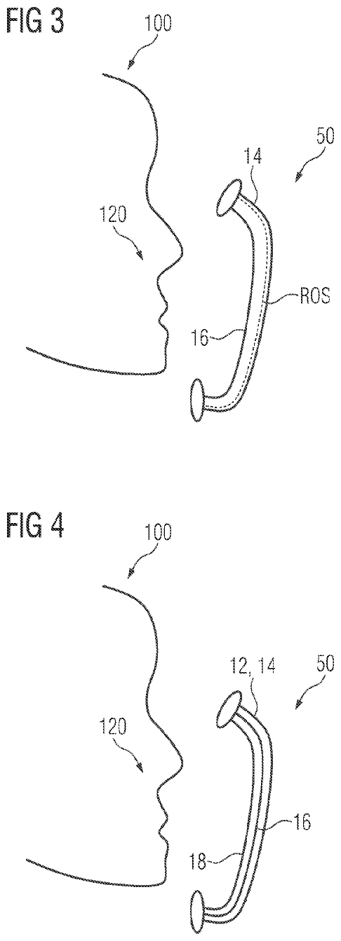

FIG. 3 shows an example mouth/nose protector 50. The mouth/nose protector 50 is often also called mouth/nose protection mask or face mask. By way of example, the mouth/nose protector 50 here has one or more nonwoven layers that may have a first region 14 and a second region 16. The first region 14 is configured to produce reactive oxygen species ROS, for example by way of a coating with silver particles that are doped with manganese(IV) oxide. The second region 16 is configured to neutralize the reactive oxygen species ROS. An elevated concentration of reactive oxygen species ROS thus does not reach a wearer 100 in the area of a mouth/nose region 120, mouth region 120 for short. This can significantly improve the compatibility of the mouth/nose protector 50 when it is worn for relatively long periods.

FIG. 4 shows an example further mouth/nose protector 50 based on nonwoven layers. A water-repellent, hydrophobic outer layer 12 is in direct contact with the ambient air. The outer layer 12 is applied here directly onto the first region 14, so that only a small amount of liquid penetrates into the mask 50. However, should infectious aerosol or droplets nevertheless get into the first region 14, then this infectious material is disinfected/neutralized by the reactive oxygen species ROS produced in the first region. A skin-compatible layer 18 that faces the wearer 100 may for example be configured as a skin-compatible polypropylene nonwoven and lies directly on the mouth/nose region 120 of the wearer 100.

In the present example, the antivirally active first region 14 is provided as part of the outer layer 12. This bactericidal and virucidal layer may consist mainly of silver and is doped with manganese dioxide (MnO₂). Silver and manganese dioxide form oxygen radicals, also known as ROS (=reactive oxygen species), from oxygen and moisture in the respiratory air. These extremely reactive oxygen species ROS can destroy proteins, lipids, RNA or DNA, of which bacteria and viruses consist, by said oxygen species entering into a chemical reaction with them, with the result that bacteria and viruses are rendered harmless.

Since excess ROS can be harmful to the throat of the wearer when inhaled and irritate the mucous membranes, an electrically conducting metal thread made of stainless steel that has a reducing effect on the ROS radicals is incorporated onto a second region 16. Stainless steel is preferred because stainless steel is harmless to the wearer 100 of the mouth/nose protector 50.

In some embodiments, the skin-compatible layer 18 may have an antibacterially and antivirally active first region 14 at least on the side that faces away from the wearer 100. This would further enhance the effect. A further second region 16, for example that is electrically conducting, may then be provided in the skin-compatible layer.

In summary, the teachings of the present disclosure relates to a fiber material composite 10 with at least a first region 14 configured to produce reactive oxygen species ROS and a second region 16 that is configured to neutralize the reactive oxygen species ROS. Furthermore, the disclosure describes a mouth/nose protector 50 and items of personal protective equipment.

### REFERENCE SYMBOLS

ROS reactive oxygen species
10 fiber material composite
12 outer layer
14 first, ROS-producing region
15 intermediate region
16 second, ROS-neutralizing region
18 skin-compatible layer
50 mouth/nose protector
100 wearer
120 mouth/nose region

What is claimed is:

1. A fiber material composite comprising:
a first region including metallic silver and manganese(IV) oxide for producing reactive oxygen species; and
a second region configured to neutralize the reactive oxygen species.

2. The fiber material composite as claimed in claim 1, wherein the first region and/or the second region include a separate ply.

3. The fiber material composite as claimed in claim 1, having an intermediate region configured as a nonwoven.

4. The fiber material composite as claimed in claim 1, wherein the first region comprises manganese dioxide-doped silver particles.

5. The fiber material composite as claimed in claim 1, wherein the first region and/or the second region a polypropylene nonwoven.

6. The fiber material composite as claimed in claim 1, wherein the second region includes an electrically conducting coating.

7. The fiber material composite as claimed in claim 1, wherein the second region comprises an electrically conducting woven fabric.

8. The fiber material composite as claimed in claim 1, wherein the second region comprises a stainless steel mesh or stainless steel grid.

9. A mouth/nose protector comprising:
a fiber material composite;
a first region including metallic silver and manganese(IV) oxide for producing reactive oxygen specifies; and
a second region configured to neutralize the reactive oxygen species.

10. The mouth/nose protector as claimed in claim 9, wherein the second region is arranged between a mouth region of a wearer of the mouth/nose protector and the first region.

11. The mouth/nose protector as claimed in claim 9, wherein the first region is arranged as the outermost layer on a side that faces away from the mouth region of a wearer of the mouth/nose protector.

12. The mouth/nose protector as claimed in claim 8, further comprising a hydrophobic layer.

13. The mouth/nose protector as claimed in claim 8, further comprising a hygroscopic layer.

14. The mouth/nose protector as claimed in claim 8, wherein the second region is part of the mouth/nose protector and the first region is designed to be exchangeable.

15. (canceled)
